(19) United States
(12) Patent Application Publication
(10) Pub. No.: US 2026/0224292 A1
(43) Pub. Date: Aug. 6, 2026

(54) Elongate Device Extraction From Intraoperative Images

(71) Applicant: INTUITIVE SURGICAL OPERATIONS, INC., Sunnyvale, CA (US)

(72) Inventors: Zhehao Hu, San Jose, CA (US); Jorge Anton Garcia, San Francisco, CA (US); Trevor W. Laing, San Jose, CA (US); Hui Zhang, Palo Alto, CA (US)

(21) Appl. No.: 19/153,241

(22) PCT Filed: Jan. 31, 2024
(86) PCT No.: PCT/US2024/013643

§ 371 Date: Aug. 1, 2025

Related U.S. Application Data
(60) Provisional application No. 63/482,749, filed on Feb. 1, 2023.

(51) Int. Cl. A61B 34/20; A61B 34/00; A61B 90/00; G06T 7/00; G06T 7/44

(52) U.S. Cl. CPC A61B 34/20; A61B 34/25; A61B 90/36; G06T 7/0012; G06T 7/44; A61B 2034/2065; A61B 2034/258

(57) ABSTRACT

Systems and methods for navigating during a medical procedure are described. Two-dimensional or three-dimensional intraoperative images are received and processed to identify, using an edge-detection algorithm, pixels corresponding to a flexible elongate device disposed within an anatomical structure. Additional operations may include: rejecting spurious edges and/or noise by applying a threshold, projecting a three-dimensional image onto a two-dimensional image, detecting contours corresponding to edges, clustering pixels corresponding to edges, and fitting detected edges or sets of detected edges (e.g., with polynomials, splines, etc.). Furthermore, a graphical user interface depicting the flexible elongate device identified based on at least some of the above operations may be displayed.

### CROSS-REFERENCE TO RELATED APPLICATIONS

This application claims priority to and the benefit of the filing date of provisional U.S. Patent Application No. 63/482,749 entitled “ELONGATE DEVICE EXTRACTION FROM INTRAOPERATIVE IMAGES,” filed on Feb. 1, 2023. The entire contents of the provisional application are hereby expressly incorporated herein by reference.

### FIELD

Disclosed examples relate to planning and/or navigating minimally invasive medical procedures and, more specifically, to localization of a flexible elongate device (e.g., a catheter) within intraoperative images.

### BACKGROUND

Minimally invasive medical techniques are intended to reduce the amount of tissue that is damaged during medical procedures, thereby reducing patient recovery time, discomfort, and harmful side effects. Such minimally invasive techniques may be performed through natural orifices in a patient anatomy or through one or more surgical incisions. Through these natural orifices or incisions, physicians may insert minimally invasive medical instruments (including surgical, diagnostic, therapeutic, and/or biopsy instruments) to reach a target tissue location. One such minimally invasive technique is to use a flexible and/or steerable elongate device, such as a flexible catheter, that can be inserted into anatomic passageways and navigated toward a region of interest within the patient anatomy.

Intraoperative imaging can greatly aid in planning and navigating a minimally invasive procedure, for example, by accurate determination of a position, orientation, and/or pose of a flexible elongate device within the patent anatomy. Identifying within intraoperative images the regions associated with the flexible elongate device often requires physician action. However, accurate and fast identification of the flexible elongate device within intraoperative images remains a challenge using current techniques.

### SUMMARY

The following presents a simplified summary of various examples described herein and is not intended to identify key or critical elements or to delineate the scope of the claims.

In some examples, a tangible, non-transitory, computer readable medium stores instructions for navigating during a medical procedure. The instructions, when executed by one or more processors, cause the one or more processors to obtain one or more images of the flexible elongate device disposed within an anatomical structure. The instructions further cause the one or more processors to determine that a plurality of pixels or voxels within the one or more images corresponds to edges of the flexible elongate device. Still further, the instructions cause the one or more processors to cause a display device to display a graphical user interface depicting the flexible elongate device based at least in part on the plurality of pixels or voxels corresponding to the edges of the flexible elongate device.

In other examples, a system for navigating during a medical procedure comprises a display device and one or more processors configured to obtain one or more images of the flexible elongate device disposed within an anatomical structure. The one or more processors are further configured to determine that a plurality of pixels or voxels within the one or more images corresponds to edges of the flexible elongate device. Still further, the one or more processors are configured to cause the display device to display a graphical user interface depicting the flexible elongate device based at least in part on the plurality of pixels or voxels corresponding to the edges of the flexible elongate device.

Still in other examples, a method of localizing a flexible elongate device disposed within an anatomical structure comprises obtaining, by one or more processors, one or more images of the flexible elongate device disposed within an anatomical structure. The method further comprises determining, by the one or more processors, that a plurality of pixels or voxels within the one or more images corresponds to edges of the flexible elongate device. Still further, the method comprises causing, by the one or more processors, a display device to display a graphical user interface depicting the flexible elongate device based at least in part on the plurality of pixels or voxels corresponding to the edges of the flexible elongate device.

It is to be understood that both the foregoing general description and the following detailed description are illustrative and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTIONS OF THE DRAWINGS

FIG. 1A depicts an example system for navigating during a medical procedure within an operating environment.

FIG. 1B is a simplified diagram of a flexible elongate device disposed within an anatomical structure.

FIG. 2A is a simplified diagram of a display depicting a portion of the flexible elongate device obscured within an anatomical structure.

FIG. 2B is a simplified diagram of the display depicting a portion of the flexible elongate device within an anatomical structure after edge detection.

FIG. 2C is a simplified diagram of the display depicting a portion of the flexible elongate device within an anatomical structure after edge detection and a thresholding operation.

FIGS. 3A-C are simplified diagrams of a portion of a flexible elongate device viewed from different angles.

FIGS. 4A-E illustrate a projection operation on a portion of a flexible elongate device after thresholding.

FIG. 5 depicts an example sequence of image processing operations to extract position, orientation, and/or pose of a flexible elongate device.

FIG. 6 is a flow diagram of a method for navigating during a medical procedure using an image processing sequence with edge detection.

FIG. 7 is a simplified diagram of a medical system according to some examples.

FIG. 8A is a simplified diagram of a medical instrument system according to some examples.

FIG. 8B is a simplified diagram of a medical instrument including a medical tool within an elongate device according to some examples.

FIGS. 9A and 9B are simplified diagrams of side views of a patient coordinate space including a medical instrument mounted on an insertion assembly according to some examples.

Examples of the present disclosure and their advantages are best understood by referring to the detailed description that follows. It should be appreciated that like reference numerals are used to identify like elements illustrated in one or more of the figures, wherein showings therein are for purposes of illustrating examples of the present disclosure and not for purposes of limiting the same.

### DETAILED DESCRIPTION

In the following description, specific details are set forth describing some examples consistent with the present disclosure. Numerous specific details are set forth in order to provide a thorough understanding of the examples. It will be apparent, however, to one skilled in the art that some examples may be practiced without some or all of these specific details. The specific examples disclosed herein are meant to be illustrative but not limiting. One skilled in the art may realize other elements that, although not specifically described here, are within the scope and the spirit of this disclosure. In addition, to avoid unnecessary repetition, one or more features shown and described in association with one example may be incorporated into other examples unless specifically described otherwise or if the one or more features would make an example non-functional. In some instances, well known methods, procedures, components, and circuits have not been described in detail so as not to unnecessarily obscure aspects of the examples.

This disclosure describes various instruments and portions of instruments in terms of their state in three-dimensional space. As used herein, the term “position” refers to the location of an object or a portion of an object in a three-dimensional space (e.g., three degrees of translational freedom along Cartesian x-, y-, and z-coordinates). As used herein, the term “orientation” refers to the rotational placement of an object or a portion of an object (e.g., one or more degrees of rotational freedom such as roll, pitch, and yaw). As used herein, the term “pose” refers to the position of an object or a portion of an object in at least one degree of translational freedom and to the orientation of that object or portion of the object in at least one degree of rotational freedom (e.g., up to six total degrees of freedom). As used herein, the term “shape” refers to a set of poses, positions, and/or orientations measured along an object. As used herein, the term “distal” refers to a position that is closer to a procedural site and the term “proximal” refers to a position that is further from the procedural site. Accordingly, the distal portion or distal end of an instrument is closer to a procedural site than a proximal portion or proximal end of the instrument when the instrument is being used as designed to perform a procedure.

This disclosure generally relates to systems and methods that facilitate user (e.g., physician) planning of, and/or user navigation during, medical procedure, such as an endoluminal medical procedure. These systems and methods can provide more precise and more automated localization of a flexible elongate device (e.g., a catheter) within intraoperative images, thereby reducing the need for manual user intervention, reducing procedure time (e.g., by removing the manual labeling operation), and improving procedure accuracy.

The system may identify, within one or more intraoperative images, the pixels and/or voxels corresponding to a catheter or other flexible elongate device. To that end, the system may perform edge detection to detect the edges of the flexible elongate device. Intraoperative images may be three-dimensional (3D) or two-dimensional (2D). For the purpose of processing according to the techniques described herein, 3D images may be decomposed into 2D image slices corresponding to spatially distributed planes or cross-sections of the patient's anatomy. The system may perform edge detection on each slice from a set of 2D slices within a three-dimensional image space. The angle (orientation) of the 2D slices may be adjusted to provide a more advantageous projection (e.g., to avoid crossings or overlaps) of the flexible elongate device. After edge detection, the system may reject spurious edges from, for example, anatomical features within the images by applying a threshold, e.g., by nulling pixels below the threshold. The thresholding may be binary, leading to two-level images of edges. In some implementations or applications, such as applications where edges may be blurred by motion, the threshold may be selected based on a histogram of the images after edge-detection. After edge detection and thresholding, 2D slices may be projected onto a single 2D image using, for example, a maximum-intensity projection. During the projection, the system may record, for each above-threshold pixel, a slice index to allow expanding the 2D projection back onto a 3D space at a later processing stage as discussed below. After constructing a two-dimensional representation of edges, the system may apply a contour detection algorithm to detect contours of groups of pixels corresponding to edges. The system may discard pixel groups with contours that have an extent (e.g., length) that falls below a threshold. Subsequently, the system may apply a clustering algorithm to remove any potential discontinuities in the representation of device edges (e.g., in the contours). The cluster including the largest number of pixels may be selected as a representation of the device contour, for example. The system may then fit the device contour with a polynomial, with splines, or with another suitable method. To determine 3D position or pose of the flexible elongate device, the system may expand the contour or the fit of the contour into 3D space using slice indices recorded in the 2D projection operation.

In some implementations or applications, the example systems and methods may be configured to process 2D intraoperative images. In these cases, the system may skip the projection and expansion (inverse-or de-projection) operations. The system may use the fit of the device contour to determine, within the image coordinate system, the position of the distal end on the flexible elongate device.

After determining the position or pose (e.g., represented as a set of pixels or voxels, or a computed fit) of the flexible elongate device, the system may generate a graphical representation of the flexible elongate device in a graphical user interface (GUI). The system may overlay the graphical representation of the flexible elongate device on or with a model of the anatomy within which the flexible elongate device is disposed. Furthermore, the system may use precise localization to better register the intraoperative imaging coordinate system with the coordinates of the flexible elongate device positioning system.

The example systems and methods may provide a number of improvements in identifying a flexible elongate device within intraoperative images and, consequently, in an overall minimally invasive medical procedure. Reducing the need for manual user intervention by automating localization of a flexible elongate device within intraoperative images may reduce overall procedure time. For example, without the presently described systems and methods, a user (e.g., physician) may rely on manually labeling of pixels and/or voxels associated with the flexible elongate device using an interactive display. Such labeling may be time consuming and/or require additional skills, e.g., that of a consulting radiologist. In the latter case, availability of a skilled user and/or delays due to interactions between an operator manipulating the flexible elongate device and the one assisting with image labeling may further lengthen procedure time. Furthermore, automating localization of a catheter or another flexible elongate device may improve procedure accuracy in several ways. The automatic extraction of pixels and/or voxels associated with the flexible elongate device may be more accurate that a human-guided extraction, resulting in more precise device localization with respect to a region of interest within patient anatomy. Additionally, by allowing the operator (e.g., physician) to focus on navigation, without having to switch to the labeling operation, the systems and methods of the present disclosure may preserve the operator attention, leading to improved procedure accuracy.

The techniques described in the disclosure may be efficiently implemented in hardware and software. The example image processing techniques are computationally efficient and can improve with improvements in imaging. That is, improvements in image resolution may directly translate in improved accuracy of the described techniques. At least in part, the improvements in accuracy directly due to improvements in image quality stem from the fact that the techniques described in this disclosure might not rely on training of machine learning models. Additionally, when accuracy can be sacrificed or when image quality is reduced, the techniques described in the disclosure can be implemented using fewer computational operations, thus making aspects of the techniques adjustable based on hardware capabilities and speed requirements.

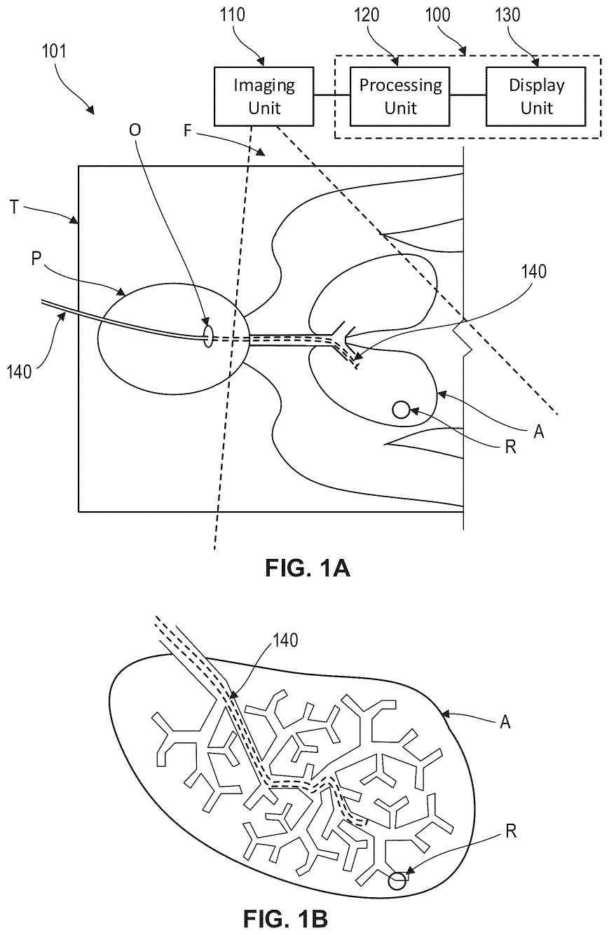

FIG. 1A depicts an example system 100 for navigating during a medical procedure within an operating environment 101. The system 100 may obtain images from a portion of the operating environment 101 disposed within a field of view F (approximately demarcated by dashed lines) of an imaging unit 110. To that end, the system 100 may be in communicative connection with the imaging unit 110. The system 100 includes a processing unit 120 and a display unit 130 in communicative connection with each other. Although in FIG. 1 the imaging unit 110 is depicted as being distinct from the system 100, in other examples, the system 100 may include the imaging unit 110. In any case, one or more processors of the processing unit 120 of the system 100 may be configured to receive images from the imaging unit 110. Throughout the disclosure, the descriptions of example operations performed by the processing unit 120 below are to be understood to be executed by the one or more processors of the processing unit 120. In some examples, the one or more processors may include hardware specifically configured (e.g., hardwired or programmable) to carry out at least a portion of the example operations described in this disclosure. Additionally or alternatively, the one or more processors may be configured to carry out at least a portion of the example operations described in this disclosure by carrying out a set of software instructions. To that end, the system may include or be communicatively connected to a tangible, non-transitory, computer readable medium. The medium may store instructions which, when executed by the processing unit 120, may perform the example operations described below. For example, the instructions may cause the processing unit 120 to perform image processing operations on the images received from the imaging unit 110. Furthermore, the instructions may cause the processing unit 120 to cause the display unit 130 to display, via a graphical user interface, information based on the processing of images received from the imaging unit (e.g., by sending the information, or sending data representing the entire graphical user interface including the information, to the display unit 130).

An operator (e.g., a physician, another medical practitioner, or a fully-automated robotic surgery system) of a medical system may use the information displayed at the display unit 130 to perform a medical procedure (e.g., endoscopy, biopsy, pharmacological treatment, and/or ablation). The medical procedure may require the operator to control a flexible elongate device 140 inserted through an orifice O into an anatomical structure A of a patient P disposed at a table T. For example, the medical procedure may include navigating the flexible elongate device 140 (indicated with solid lines outside and dashed lines inside the patient P) toward a region of interest R within the anatomical structure A with the aid of information displayed at the display unit 130. The region R, for example, may be a designated procedure site for examination, biopsy, surgery, or another treatment.

FIG. 1B is a simplified diagram of the flexible elongate device disposed within the anatomical structure A. FIG. 1B is included to give an expanded and more detailed view of a portion of the operating environment 101 disposed within the field of view F. The anatomical structure A may be a lung of the patient P. The flexible elongate device 140 may be inserted into and navigated by the operator toward the region R, for example, for the purpose of investigating or treating a pathology in the region R. The techniques described in the present disclosure can facilitate the navigation process by generating and displaying timely and accurate sensing (e.g., imaging) and detection (e.g., identification) of the device 140.

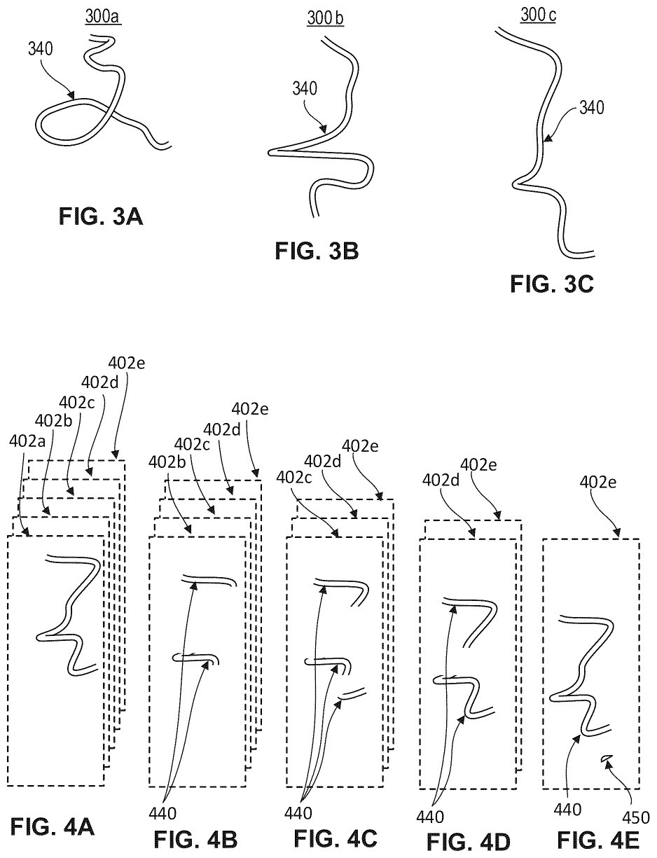

Returning to FIG. 1A, the images generated by the imaging unit 110 may be two-dimensional (2D) or volumetric, three-dimensional (3D) images. 3D images may include a collection of voxels on a regular grid, point clouds, or a set of 2D slices that collectively represent a three-dimensional volume. Each 2D slice of a 3D image or a whole 2D image may include a collection of pixels. Each pixel may represent a point on a planar or curved surface within the field of view F. One or more processors may be configured to transform 3D images into a set of 2D images, convert 3D images from point clouds to voxels of a regular grid, or re-grid or interpolate from one 2D or 3D grid to another one, e.g., of higher or lower resolution or a different orientation. Generating 2D slices with alternative orientations from a 3D image is discussed in more detail with reference to FIGS. 3A-C. The imaging unit 120 and/or the processing unit 110 may include at least some of the one or more processors configured to perform the operations described above.

The processing unit 120 of the system 100 may obtain one or more images of the flexible elongate device 140 disposed within the anatomical structure A. In some examples, the processing unit 120 may request the imaging unit 110 to generate the one or more images. In other examples, the imaging unit 110 may continuously generate (and, possibly, buffer or store) images of the scene within the field of view F. Generally, the processing unit 120 may cooperate with the imaging unit 110 to obtain images in the appropriate format for further processing.

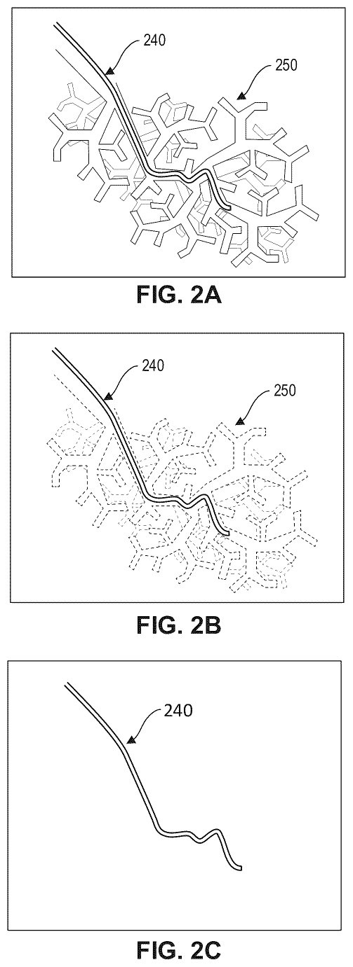

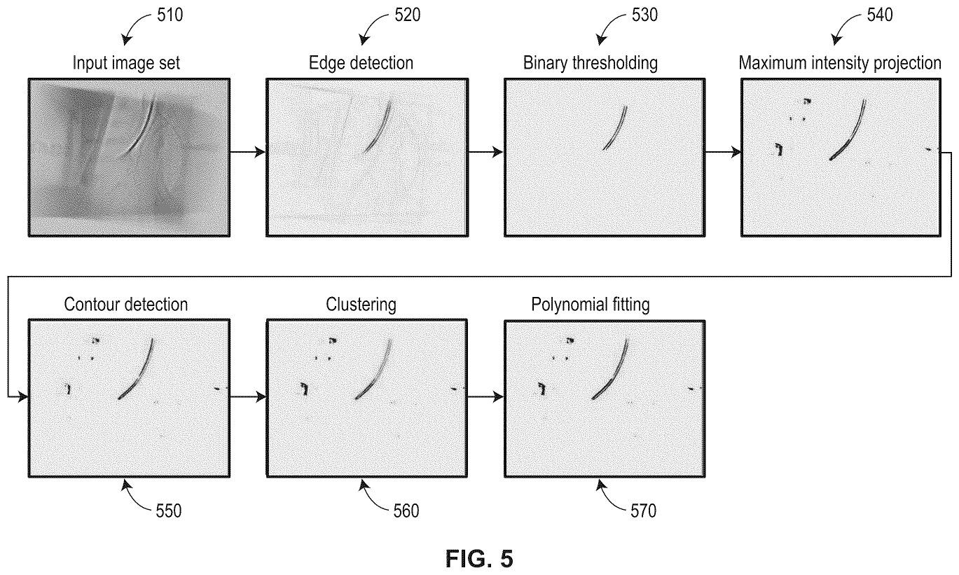

The processing unit 120 of the system 100 may perform one or more image processing operations on the images obtained from the imaging unit 110. As described in more detail, for example, with reference to FIGS. 2A-C and FIG. 5, the processing unit 120 may determine that a particular plurality of pixels or voxels within the one or more images corresponds to edges of the flexible elongate device 140. To that end, the processing unit 120 may be configured to execute one or more image processing algorithms for edge detection as described below. The processing unit 120 may also be configured to cause the display device 130 to display a graphical user interface depicting the flexible elongate device 140 based at least in part on the plurality of pixels or voxels corresponding to the edges of the flexible elongate device 140. For example, the processing unit 120 may cause the display device 130 to render one or more images of the anatomical structure A along with the highlighted portion of the image corresponding to the device 140. Furthermore, the processing unit 120 may cause the display device 130 to display information indicative of position, orientation, and/or pose of the device 140. Extracting, based on the detected edges, pixels and/or voxels corresponding to the flexible elongate device 140, and/or based on information indicative of position, orientation, and/or pose of the device 140, may include a number of image processing operations described, for example, with reference to FIG. 5. Among other operations, the processing unit 120 may project a portion of a 3D image (e.g., a stack of 2D images) onto a single 2D image, as discussed in detail with reference to FIGS. 4A-E. The processing unit 120 may perform additional processing on the projected image and, at a later stage, may reverse the projection, e.g., restore, expand, or de-project a processed 2D image back to a 3D image.

FIGS. 2A-C illustrate example image processing operations that the processing unit 120 of the system 100 may perform and the example information that the processing unit 120 may cause the display unit 130 to display. FIG. 2A is a simplified diagram of a display 230 (e.g., portion of the display unit 130) depicting a portion of a flexible elongate device 240 (e.g., device 140) obscured within an anatomical structure 250 (e.g., the airways or bronchi of the anatomical structure A). In the context of FIGS. 2A-C, the device 240 may correspond to the portion of an image depicting the device, as displayed by the display 230. Analogously, the anatomical structure 250 may correspond to the portion of the image depicting the anatomical structure, as displayed on the display 230.

The display 230 may be a portion of a monitor, a mobile device, a virtual reality (VR) headset, or any other suitable display device. Furthermore, a user or operator may interact with the display 230 using a touch-screen of the display 230, a keyboard, a joystick, a glove, an inertial motion unit (e.g., integrated into a headset), or any other suitable user input device.

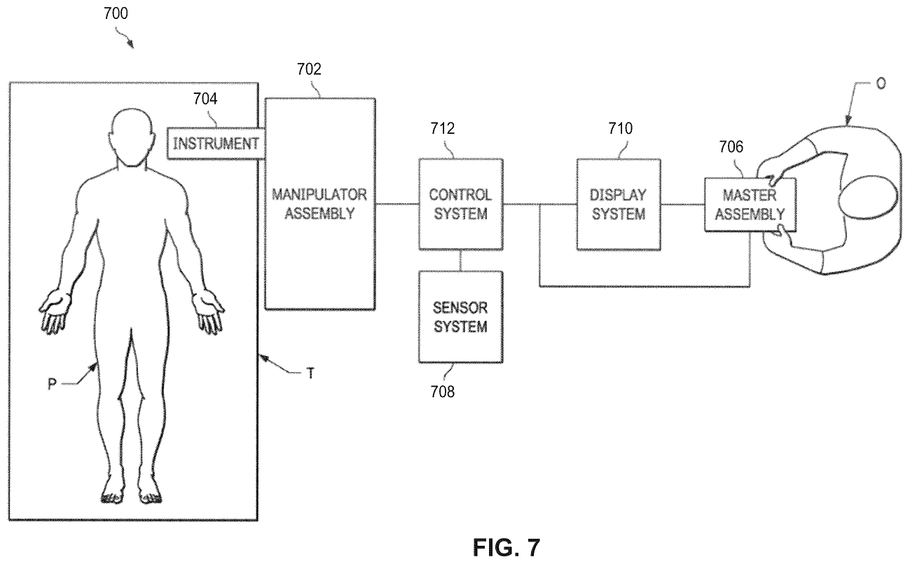

Using an image displayed in FIG. 2A by the display 230, the operator may have difficulty identifying the portion of the image depicting the flexible elongate device 240. Even if the operator is capable of identifying the pixels and/or voxels associated with the device 240, the system (e.g., system 100) may be unable to generate position, orientation, and/or pose information for the device 240 without effortful and time-consuming operator input. As will be described in further detail below, the system may perform an image processing operation, such as an edge detection operation, to facilitate distinguishing the device 240 from the anatomical structure 250. In some examples, a tip portion (distal portion, as described below with reference to FIGS. 7-9B) may be of particular importance. The display 230 may be configured to display, based on the techniques of this disclosure, only the tip portion of the device 240 disposed within the anatomy 250.

FIG. 2B is a simplified diagram of the display 230 depicting a portion of the flexible elongate device 240 within the anatomical structure 250 after edge detection. The system (e.g., system 100) may perform edge detection to facilitate distinguishing (by the system 100 or an operator) the device 240 from the anatomical structure 250. For example, a processing unit (e.g., processing unit 110) of the system may apply an edge-enhancing digital filter as a convolution in the image domain or a weighted summation in the Fourier domain. In some examples, the system may implement a digital convolution along each of two dimensions of a 2D image using a suitable kernel (e.g., Roberts cross, Prewitt, Sobel, Laplacian, etc.) to estimate components of a gradient in a single-channel image. The system may implement a smoothing (e.g., Gaussian) or any other suitable noise-reducing filter prior to convolutions with edge-detecting kernels. In the case of separable gradient-estimating kernels (e.g., Prewitt, Sobel, etc.), the system may estimate the total gradient for each single-channel 2D image as a square root of the sum of the squares of the component gradient estimates. In some examples, the system may perform a two-dimensional convolution with two-dimensional kernels (e.g., Laplacian or Laplacian of Gaussian) of any suitable size (e.g., 3×3, 5×5, 7×7, etc.) to detect edges. The system may further add non-local-maximum suppression, thresholding, and/or hysteresis analysis, as in, for example, Canny edge detection. Additionally or alternatively, the system may use other edge-detecting or edge-preserving filters such as median, wavelet, Gabor, or Log-Gabor filters. Furthermore, the system may use machine learning (ML) methods for edge detection, such as holistically-nested edge detection (HED). In multi-channel images (e.g., images with color channels), the system may perform edge detection for each channel and combine the channel estimates. Additionally or alternatively, the system may combine the channels (e.g., compute an estimate of brightness or another suitable metric) and perform edge detection on a single channel image.

The result of performing edge detection is illustrated in FIG. 2B. The edges of the flexible elongate device 240 may appear prominently in the image (e.g., higher values in the resulting image array), while much the anatomical structure 250 may be suppressed (e.g., have lower values). Though the flexible elongate device 240 may no longer be obscured by the anatomical structure 250 in FIG. 2B to an eye of the operator, the system may perform additional processing to extract the pixels and/or voxels associated with the flexible elongate device 240. For example, the pixels in FIG. 2B that have high values indicative of edges (e.g., the edges of the flexible elongate device 240) may be separated or extracted from the rest of the image by a thresholding operation, as illustrated in FIG. 2C.

FIG. 2C is a simplified diagram of the display 230 depicting a portion of the flexible elongate device 240 within the anatomical structure 250 after edge detection (as described with reference to FIG. 2B) and a thresholding operation. The system (e.g., system 100) generates a post-threshold image (e.g., the image schematically depicted in FIG. 2C) by assigning a high value (e.g., 1, 100, 255, etc.) to the pixels with values at or above a threshold and a low value (e.g., 0) to the pixels with values below the threshold. In some examples, the system assigns a color value to pixels above the threshold to display in images. Regardless of the images for display to the user, the system may generate a thresholded image array based on binary thresholding. The system may process the thresholded array to extract additional information (e.g., position, orientation, pose, etc.) associated with the flexible elongate device 240.

In some examples, the system (e.g., system 100) automatically generates a threshold value after edge detection. For example, the system may generate the threshold based on a histogram of the image array after edge detection. The system may automatically select histogram bin number and sizes or use pre-set bins for the histogram. The system may select a threshold value suitably below a peak value of a mode associated with edges. Additionally or alternatively, the system may use other thresholding techniques based on clustering, entropy, etc. Furthermore, the system may select different threshold levels for different regions of the image. In other examples, the system prompts a user or operator for input in selecting an appropriate threshold level.

It should be noted that the system need not display the images in FIGS. 2A-C and the images may simply illustrate image processing operations, the results of which are not directly displayed to the operator. Instead, the system may further process the images to display suitable information on the display 230, as described below.

When analyzing 3D images, the system may select an orientation of 2D slices prior to thresholding. In other examples, the system performs the thresholding operation on one set of slices and, subsequently, slices (e.g., using re-gridding, interpolation, etc.) the 3D image in a different orientation to generate a different set of slices for further processing. The system may perform another set of thresholding operations after re-slicing. The system may select, automatically or with user or operator input, a suitable orientation of slices that facilitates extracting information associated with a flexible elongate device (e.g., device 240) disposed within an anatomical structure (e.g., structure 250).

FIGS. 3A-C are simplified diagrams of a portion of a flexible elongate device 340 (e.g., devices 140, 240) viewed from different angles. In a sense, views 300a-c represent 2D projections of 3D images of the device 340. The pose of the device 340 can be assumed to be the same in the three views 300a-c. Viewed from the angle of FIG. 3A, however, the projection of the flexible elongate device 340 has a crossing, while in the views of FIGS. 3B-C, the crossing is eliminated. The system (e.g., system 100) may select a view (e.g., view 300b-c) that avoids crossings for further processing. Furthermore, the system and/or operator may select a view with maximum extent, minimized curvature, etc. Certain properties of projections from different views may facilitate further image processing operations.

FIGS. 4A-E illustrate a projection operation on a portion of a flexible elongate device after thresholding. A system (e.g., system 100) may perform the projection operation to aggregate pixels associate with a flexible elongate device 440 (e.g., devices 140, 240, 340) from multiple 2D slices onto a single 2D image. For the sake of illustrating the operation, pixels or voxels associated with the flexible elongate device are disposed at or between five bounding planes 402a-e. The five bounding planes 402a-e may represent four image slices: the first (bound by the planes 402a and b), the second (bound by the planes 402 b and c), the third (bound by the 402c and d), and the fourth (bound by the planes 402d and e). The values represented by dark regions in FIGS. 4A-E between the planes 402a and e may be the high binary values generated by the thresolding operation, with the low binary values elsewhere. In FIG. 4A, all of the dark regions are obscured. Removing the plane 402a, as illustrated in FIG. 4B, exposes the above-threshold regions of the first image slice. Removing the planes 402a and b, as illustrated in FIG. 4C, exposes the above-threshold regions of the first and second slices, now aggregated into a single 2D image. In operations aggregating binary values, an overlap of high-value regions in distinct slices may be represented by a high value in the projection. The implementation of the projection may be thought of as a maximum intensity projection (MIP) for two-level images. The MIP for two-level images may also be thought of as a logical OR operation on a set of corresponding pixels (pixels with the same x-and y-coordinates or indices) from multiple slices. In some examples, the system may perform a MIP before thresholding, selecting the brightest pixel from each slice. The system may use the resulting projection to, for example, select an appropriate threshold level.

Referring now to FIG. 4D, where planes 402a-c have been removed, the dark region represents a union of the dark regions in the first three slices. Finally, removing the plane 402d exposes, in FIG. 4E, the union of all the dark regions between the planes 402a and e. As illustrated in FIG. 4E, the pixel values associated with edges of the flexible elongate device 440 may form contiguous contours. A spurious feature 450, either an aspect of the anatomy or an artifact, may be distinguishable (by the disjoint collection of pixels) from the pixels associated with the flexible elongate device 440, as described below with reference to FIG. 5.

The system may be configured to reverse the projection operation (e.g., MIP) described with reference to FIGS. 4A-E. For example, the system may record x-and y-indices of the projected values from each slice and/or record a slice index (or multiple slice indices) for each high (e.g., above threshold) pixel value in the projection. With this recorded information, the system may expand or de-project a 2D projection into a 3D image.

FIG. 5 illustrates an example sequence of image processing operations 510-570 to extract position, location and/or pose of a flexible elongate device. The processing operations 510-570 may be performed, as described throughout the disclosure, by one or more processors (e.g., included in the processing unit 120) of an example system (e.g., system 100). Each operation 510-570 in FIG. 5 is represented by an accompanying image. The system may display or not, generate, or display aspects of, those accompanying images (e.g., using the display unit 130). Regardless of what images the system generates and/or displays, the accompanying images help illustrate the image processing operations that may be performed by the system. In different example implementations, the system may implement the operations 510-570 in a different order or may omit some of the operations 510-570 altogether.

In operation 510, the example system may obtain an input image set (e.g., generated by the imaging unit 110). For example, an imaging unit (e.g., the imaging unit 110) may generate 2D images, such as, for example, 2D fluoroscopic images, or 3D images, such as, for example, cone beam computer tomography (CBCT), or tomosynthesis images. The image accompanying operation 510, analogously to the image in FIG. 2A, includes many features of the anatomy (e.g., lung airways, bony structures such as ribs, etc.) along with the features associated with a flexible elongate device.

In operation 520, the example system may apply an edge detection technique. For example, the system may use a Sobel edge detection technique. The Sobel edge detection technique estimates image intensity gradient magnitude by performing a convolution with a pair of convolution kernels designed to respond maximally to edges running vertically and horizontally relative to the pixel grid. An approximate magnitude of a total gradient is the root sum of squares of the gradients in each of the two directions. The Sobel edge detection technique combines effectiveness and suitable level of computational complexity. The system may additionally or alternatively use other edge detection techniques, such as Laplacian edge detection, Canny edge detection, ML-based edge detection, etc., including any of the edge detection techniques described above with reference to FIGS. 2A-B. The image accompanying operation 520, analogously to the image in FIG. 2B, includes the enhanced edges among the suppressed anatomical features.

In operation 530, the example system may apply a thresholding operation, nulling out pixels with values below a threshold and extracting the edges of the flexible elongate device, as described above with reference to FIG. 2C. The smaller value pixels may be indicative of lack of edges, comparatively blurry edges of anatomical structures, and/or noise. For example, the image accompanying operation 520 includes a number of faint edges and some dark gray areas due to noise, both of which are black (zeroed) in the image accompanying operation 530.

In operation 540, the example system may perform a projection (e.g., a MIP) as described above with reference to FIGS. 4A-E for image sets having multiple 2D slices or other 3D image data sets. In the case of a MIP on a binary thresholded image, the projection can be thought of as a visualization plane illuminated by light sources, corresponding to the above-threshold pixels, emitting in the direction orthogonal to the visualization plane.

In operation 550, the system may process the image generated after the projection of operation 540 for 3D images or thresholding operation 530 for 2D images by applying a contour detection algorithm. As discussed with reference to FIG. 4E, a projected portion of a flexible elongate device is likely to form long contours, while spurious features may not. Thus, the system may detect, in the post-projection image, contours of various lengths and remove regions enclosed by contours of length below a threshold length. The threshold length may be pre-determined in the system configuration (e.g., hard-coded into software or selected at an onset of a medical procedure), selected based on an input from an operator or user of the system, and/or determined automatically by the system based on image properties or properties of the contours.

In operation 560, the example system may perform a clustering algorithm to cluster contours or regions enclosed within contours. The clustering operation may resolve discontinuities in detected edges and/or contours due to crossings in a projection of a flexible elongate device (e.g., as in FIG. 3A). The system may use a density-based spatial clustering application in noise (DBSCAN) algorithm and/or other clustering algorithms, such as k-means, k-nearest-neighbor, hierarchical, etc. The example system may use the DBSCAN algorithm to obviate the need of determining a priori the number of clusters. After completing clustering, the example system may select the largest cluster (e.g., the cluster with the largest number of contour points) as the one associated with the flexible elongate device. In some example implementations, the system computes a metric confidence associated with the clustering and may prompt a user to resolve, for example, whether two clusters should be merged or a single cluster should be separated. In some example implementations, the system may change or select a threshold in the thresholding operation 530 based on the number and properties of clusters. That is, there may be a feedback loop connecting operation 560 back to operation 530. Additionally or alternatively, the system may use the outcome of the clustering algorithm to select a view angle of the MIP or another projection algorithm, as discussed with reference to FIGS. 3A-C.

In operation 570, the example system may fit a polynomial to the extracted edge contours associated with a flexible elongate device. The system may use a total least squares, also known as orthogonal least squares method, minimizing the average square distance between the fitted polynomial and the binary pixel values of the extracted edge contours. Alternatively, the system may use a standard least squares polynomial fit, after suitably choosing orientations of independent and dependent variable axes. Still alternatively, the system may fit the extracted edge contours with splines. In some examples, the system is configured perform a regularized regression to obtain a fit. To that end, an objective function of regression may trade-off quality of fit (e.g., aggregate least squares residual) with another parameter, such as curvature, smoothness in curvature variation, etc. The result of the fit may me thought of as a center line curve representing the flexible elongate device, balancing the distance between opposing edges.

For 3D images, the system may be configured to de-project the polynomial fit into three dimensions using the information recorded during the projection operation, as discussed above with reference to FIGS. 4A-E. For example, the system may trace each point on the center line fit along the projection direction to the slice of the nearest pixels with the maximum signal intensity. In other example implementations, the system performs de-projection prior to the fitting operation and fitting the center line curve in 3D (e.g., as a parametric polynomial).

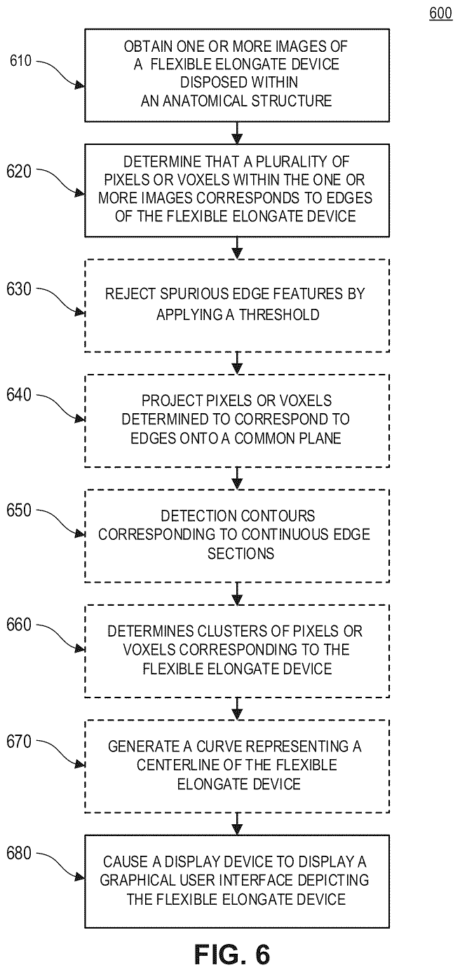

FIG. 6 is a flow diagram of a method 600 for navigating during a medical procedure using an image processing sequence with edge detection. The method 600 may be implemented using, for example, the system 100 as described above. Generally, the method 600 facilitates extracting position, orientation, pose, and/or any other suitable information descriptive of a flexible elongate device (e.g., device 140, 240, 340, 440) disposed within an anatomical structure (e.g., lungs, gastrointestinal tract, structures of the renal system, etc.).

At block 610, the method 600 includes obtaining, by a system (e.g., the system 100), one or more images of the flexible elongate device disposed within the anatomical structure. An imaging unit (e.g., imaging unit 110) may generate the images obtained by the system by infrared, terahertz, X-ray, computer-aided tomography (CAT) (e.g., CBCT), positron-emission tomography (PET), optical coherence tomography (OCT), magnetic resonance imaging (MRI), fluoroscopy, sonography, or any other suitable modality. The system may obtain images from any suitable number of imaging units and from any suitable combination of modalities. At least one imaging unit may be included in the system.

At block 620, the method 600 includes determining that a plurality of pixels or voxels within the one or more images corresponds to edges of the flexible elongate device. To that end, the system implementing the method 600 may apply an edge-detection algorithm to the one or more obtained images and use the enhanced edges to extract information about the flexible elongate device. Additionally or alternatively, the system may apply an edge-detection algorithm to new images generated by the system based at least in part on the obtained images. That is, the system may apply suitable pre-processing operations (e.g., de-noising, motion compensation, etc.) to the obtained images prior to edge detection. The method 600 may be based on the assumption that the flexible elongate device has well-defined edges within the images obtained or generated (e.g., through pre-processing of obtained images) by the system.

The method may include convolutional (e.g., with Sobel kernels), Fourier-domain, wavelet, or nonlinear filtering. Additional techniques, such as one or more of blocks 630-650, may enhance the accuracy of determining the pixels or voxels that correspond to the edges of the flexible elongate device.

Blocks 630-670 are optional blocks that may be included in the method 600 to facilitate extracting position, orientation, pose, and or any other suitable information descriptive of the flexible elongate device disposed within the anatomical structure. In some examples, one or more of the blocks 630-670 are included in the block 620.

At block 630, the method 600 includes rejecting spurious edge featured by applying a threshold, as described, for example, with reference to FIGS. 2A-C and operation 530 of FIG. 5. In some examples, the method includes prompting an operator, via a graphical user interface, to select a suitable threshold. In other examples, the system selects a suitable threshold value automatically. The system may use image histogram analysis or analysis of clustering or contour detection to select a suitable threshold.

At block 640, the method 600 includes projecting pixels or voxels determined to correspond to edges onto a common plane, as described, for example, with reference to FIGS. 4A-E and operation 540 of FIG. 5. The method may include block 640 when processing 3D images. The system may use maximum-intensity projection or any other suitable projection method to aggregate a two-dimensional representation of edge features from 3D images. The method 600 may include de-projecting or expanding 2D images back into 3D, as described above.

At block 650, the example method 600 includes detecting contours corresponding to continuous edge section, as described, for example, with reference to operation 550 of FIG. 5. Detecting contours corresponding to continuous edge sections may include rejecting (e.g., suppressing, nulling, etc.) groups of pixels bound by short contours (e.g., contours below a threshold length).

At block 660, the method 600 includes determining clusters of pixels or voxels corresponding to the flexible elongate device, as described, for example, with reference to operation 560 of FIG. 5. The method 600 may include DBSCAN clustering or other suitable clustering algorithms. The clustering operation may join together continuous edge sections based on detected contours.

At block 670, the method 600 includes generating a curve representing a centerline of the flexible elongate device. To generate the centerline curve, the method 600 may include polynomial fitting, spline fitting, or any other suitable technique, as described, for example, with reference to operation 570 of FIG. 5.

At block 680, the method 600 includes causing a display device (e.g., device 130) to display a graphical user interface depicting the flexible elongate device based at least in part on the plurality of pixels or voxels corresponding to the edges of the flexible elongate device. In some examples, the method 600 includes displaying one or more images corresponding to operations of the method 600, as illustrated in FIGS. 2A-C and FIG. 5. The method 600 may include displaying the flexible elongate device in color or as a bright feature overlaid with anatomical structure in a different color or less bright images. The method 600 may include displaying the center line fit (e.g., generated at block 670) or a shape based on the center line fit in the GUI of the display. In some examples, the method may include displaying only a tip portion of the flexible elongate device to avoid display clutter. The tip portion may be determined based on a distal portion (as explained below) of the center line fit.

FIGS. 7-9B depict diagrams of a medical system that may be used for manipulating a medical instrument that includes a flexible elongate device according to any of the methods and systems described above, in some examples. For example, each reference above to the “system” may refer to a system (e.g., system 700) discussed below, or to a subsystem thereof.

FIG. 7 is a simplified diagram of a medical system 700 according to some examples. The medical system 700 may include at least portions of the system 100 described with reference to FIG. 1. The medical system 700 may be suitable for use in, for example, surgical, diagnostic (e.g., biopsy), or therapeutic (e.g., ablation, electroporation, etc.) procedures. While some examples are provided herein with respect to such procedures, any reference to medical or surgical instruments and medical or surgical methods is non-limiting. The systems, instruments, and methods described herein may be used for animals, human cadavers, animal cadavers, portions of human or animal anatomy, non-surgical diagnosis, as well as for industrial systems, general or special purpose robotic systems, general or special purpose teleoperational systems, or robotic medical systems.

As shown in FIG. 7, medical system 700 may include a manipulator assembly 702 that controls the operation of a medical instrument 704 in performing various procedures on a patient (e.g., patient P on table T, as in FIG. 1). The medical instrument 704 may include the flexible elongated device 140 of FIG. 1 and/or devices 240, 340, 440 of FIGS. 2A-4E. Medical instrument 704 may extend into an internal site within the body of patient P via an opening in the body of patient P. The manipulator assembly 702 may be teleoperated, non-teleoperated, or a hybrid teleoperated and non-teleoperated assembly with one or more degrees of freedom of motion that may be motorized and/or one or more degrees of freedom of motion that may be non-motorized (e.g., manually operated). The manipulator assembly 702 may be mounted to and/or positioned near patient table T. A master assembly 706 allows an operator O (e.g., a surgeon, a clinician, a physician, or other user, as described above) to control the manipulator assembly 702. In some examples, the master assembly 706 allows the operator O to view the procedural site or other graphical or informational displays. In some examples, the manipulator assembly 702 may be excluded from the medical system 700 and the instrument 704 may be controlled directly by the operator O. In some examples, the manipulator assembly 702 may be manually controlled by the operator O. Direct operator control may include various handles and operator interfaces for hand-held operation of the instrument 704.

The master assembly 706 may be located at a surgeon's console which is in proximity to (e.g., in the same room as) the patient table T on which patient P is located, such as at the side of the patient table T. In some examples, the master assembly 706 is remote from the patient table T, such as in in a different room or a different building from the patient table T. The master assembly 706 may include one or more control devices for controlling the manipulator assembly 702. The control devices may include any number of a variety of input devices, such as joysticks, trackballs, scroll wheels, directional pads, buttons, data gloves, trigger-guns, hand-operated controllers, voice recognition devices, motion or presence sensors, and/or the like.

The manipulator assembly 702 supports the medical instrument 704 and may include a kinematic structure of links that provide a set-up structure. The links may include one or more non-servo controlled links (e.g., one or more links that may be manually positioned and locked in place) and/or one or more servo controlled links (e.g., one or more links that may be controlled in response to commands, such as from a control system 712). The manipulator assembly 702 may include a plurality of actuators (e.g., motors) that drive inputs on the medical instrument 704 in response to commands, such as from the control system 712. The actuators may include drive systems that move the medical instrument 704 in various ways when coupled to the medical instrument 704. For example, one or more actuators may advance medical instrument 704 into a naturally or surgically created anatomic orifice. Actuators may control articulation of the medical instrument 704, such as by moving the distal end (or any other portion) of medical instrument 704 in multiple degrees of freedom. These degrees of freedom may include three degrees of linear motion (e.g., linear motion along the X, Y, Z Cartesian axes) and in three degrees of rotational motion (e.g., rotation about the X, Y, Z Cartesian axes). One or more actuators may control rotation of the medical instrument about a longitudinal axis. Actuators can also be used to move an articulable end effector of medical instrument 704, such as for grasping tissue in the jaws of a biopsy device and/or the like, or may be used to move or otherwise control tools (e.g., imaging tools, ablation tools, biopsy tools, electroporation tools, etc.) that are inserted within the medical instrument 704.

The control system 704 may include at least portions of the processing unit 120. Additionally or alternatively, the control system 704 may be in communicative connection with the processing unit 120. In some examples, the output of the processing unit 120 according to the techniques described above may cause the control system 704 to autonomously (without input from the operator O) control certain movements of the instrument 104.

The medical system 700 may include a sensor system 708 with one or more sub-systems for receiving information about the manipulator assembly 702 and/or the medical instrument 704. Such sub-systems may include a position sensor system (e.g., that uses electromagnetic (EM) sensors or other types of sensors that detect position or location); a shape sensor system for determining the position, orientation, speed, velocity, pose, and/or shape of a distal end and/or of one or more segments along a flexible body of the medical instrument 704; a visualization system (e.g., using a color imaging device, an infrared imaging device, an ultrasound imaging device, an x-ray imaging device, a fluoroscopic imaging device, a computed tomography (CT) imaging device, a magnetic resonance imaging (MRI) imaging device, or some other type of imaging device) for capturing images, such as from the distal end of medical instrument 704 or from some other location; and/or actuator position sensors such as resolvers, encoders, potentiometers, and the like that describe the rotation and/or orientation of the actuators controlling the medical instrument 704. The sensor system 708 may include at least portions of the imaging unit 110 of FIG. 1.

The medical system 700 may include a display system 710 for displaying an image or representation of the procedural site and the medical instrument 704. Display system 710 and master assembly 706 may be oriented so physician O can control medical instrument 704 and master assembly 706 with the perception of telepresence. The display system 710 may include at least portions of the display unit 140.

In some examples, the medical instrument 704 may include a visualization system, which may include an image capture assembly that records a concurrent or real-time image of a procedural site and provides the image to the operator O through one or more displays of display system 710. The image capture assembly may include various types of imaging devices (e.g., imaging unit 110). The concurrent image may be, for example, a two-dimensional image or a three-dimensional image captured by an endoscope positioned within the anatomical procedural site. In some examples, the visualization system may include endoscopic components that may be integrally or removably coupled to medical instrument 704. Additionally or alternatively, a separate endoscope, attached to a separate manipulator assembly, may be used with medical instrument 704 to image the procedural site. The visualization system may be implemented as hardware, firmware, software or a combination thereof which interact with or are otherwise executed by one or more computer processors, such as of the control system 712.

Display system 710 may also display an image of the procedural site and medical instruments, which may be captured by the visualization system. In some examples, the medical system 700 provides a perception of telepresence to the operator O. For example, images captured by an imaging device at a distal portion of the medical instrument 704 may be presented by the display system 710 to provide the perception of being at the distal portion of the medical instrument 704 to the operator O. The input to the master assembly 706 provided by the operator O may move the distal portion of the medical instrument 704 in a manner that corresponds with the nature of the input (e.g., distal tip turns right when a trackball is rolled to the right) and results in corresponding change to the perspective of the images captured by the imaging device at the distal portion of the medical instrument 704. As such, the perception of telepresence for the operator O is maintained as the medical instrument 704 is moved using the master assembly 706. The operator O can manipulate the medical instrument 704 and hand controls of the master assembly 706 as if viewing the workspace in substantially true presence, simulating the experience of an operator that is physically manipulating the medical instrument 704 from within the patient anatomy.

In some examples, the display system 710 may present virtual images of a procedural site that are created using image data recorded pre-operatively (e.g., prior to the procedure performed by the medical instrument system 200) or intra-operatively (e.g., concurrent with the procedure performed by the medical instrument system 200), such as image data created using computed tomography (CT), magnetic resonance imaging (MRI), positron emission tomography (PET), fluoroscopy, thermography, ultrasound, optical coherence tomography (OCT), thermal imaging, impedance imaging, laser imaging, nanotube X-ray imaging, and/or the like. The virtual images may include two-dimensional, three-dimensional, or higher-dimensional (e.g., including, for example, time based or velocity-based information) images. In some examples, one or more models are created from pre-operative or intra-operative image data sets and the virtual images are generated using the one or more models.

In some examples, for purposes of imaged guided medical procedures, display system 710 may display a virtual image that is generated based on tracking the location of medical instrument 704. For example, the tracked location of the medical instrument 704 may be registered (e.g., dynamically referenced) with the model generated using the pre-operative or intra-operative images, with different portions of the model correspond with different locations of the patient anatomy. As the medical instrument 704 moves through the patient anatomy, the registration is used to determine portions of the model corresponding with the location and/or perspective of the medical instrument 704 and virtual images are generated using the determined portions of the model. This may be done to present the operator O with virtual images of the internal procedural site from viewpoints of medical instrument 704 that correspond with the tracked locations of the medical instrument 704.

The display system 710 may include the display unit 140 and may display images including the position, orientation, and/or pose of the medical instrument 704 according to the techniques described above with reference to FIGS. 1A-6.

The medical system 700 may also include the control system 712, which may include processing circuitry (e.g., the processing unit 120) that implements the some or all of the methods or functionality discussed herein. The control system 712 may include at least one memory and at least one processor for controlling the operations of the manipulator assembly 702, the medical instrument 704, the master assembly 706, the sensor system 708, and/or the display system 710. Control system 712 may include instructions (e.g., a non-transitory machine-readable medium storing the instructions) that when executed by the at least one processor, configures the one or more processors to implement some or all of the methods or functionality discussed herein. While the control system 712 is shown as a single block in FIG. 7, the control system 712 may include two or more separate data processing circuits with one portion of the processing being performed at the manipulator assembly 702, another portion of the processing being performed at the master assembly 706, and/or the like. In some examples, the control system 712 may include other types of processing circuitry, such as application-specific integrated circuits (ASICs) and/or field-programmable gate array (FPGAs). The control system 712 may be implemented using hardware, firmware, software, or a combination thereof.

In some examples, the control system 712 may receive feedback from the medical instrument 704, such as force and/or torque feedback. Responsive to the feedback, the control system 712 may transmit signals to the master assembly 706. In some examples, the control system 712 may transmit signals instructing one or more actuators of the manipulator assembly 702 to move the medical instrument 704. In some examples, the control system 712 may transmit informational displays regarding the feedback to the display system 710 for presentation or perform other types of actions based on the feedback.

The control system 712 may include a virtual visualization system to provide navigation assistance to operator O when controlling the medical instrument 704 during an image-guided medical procedure. Virtual navigation using the virtual visualization system may be based upon an acquired pre-operative or intra-operative dataset of anatomic passageways of the patient P. The control system 712 or a separate computing device may convert the recorded images, using programmed instructions alone or in combination with operator inputs, into a model of the patient anatomy. The model may include a segmented two-dimensional or three-dimensional composite representation of a partial or an entire anatomic organ or anatomic region. An image data set may be associated with the composite representation. The virtual visualization system may obtain sensor data from the sensor system 708 that is used to compute an (e.g., approximate) location of the medical instrument 704 with respect to the anatomy of patient P. The sensor system 708 may be used to register and display the medical instrument 704 together with the pre-operatively or intra-operatively recorded images. For example, PCT Publication WO 2016/191298 (published Dec. 1, 2016 and titled “Systems and Methods of Registration for Image Guided Surgery”), which is incorporated by reference herein in its entirety, discloses example systems.

During a virtual navigation procedure, the sensor system 708 may be used to compute the (e.g., approximate) location of the medical instrument 704 with respect to the anatomy of patient P. The location can be used to produce both macro-level (e.g., external) tracking images of the anatomy of patient P and virtual internal images of the anatomy of patient P. The system may include one or more electromagnetic (EM) sensors, fiber optic sensors, and/or other sensors to register and display a medical instrument together with pre-operatively recorded medical images. For example, U.S. Pat. No. 8,900,131 (filed May 13, 2011 and titled “Medical System Providing Dynamic Registration of a Model of an Anatomic Structure for Image-Guided Surgery”), which is incorporated by reference herein in its entirety, discloses example systems.

Medical system 700 may further include operations and support systems (not shown) such as illumination systems, steering control systems, irrigation systems, and/or suction systems. In some examples, the medical system 700 may include more than one manipulator assembly and/or more than one master assembly. The exact number of manipulator assemblies may depend on the medical procedure and space constraints within the procedural room, among other factors. Multiple master assemblies may be co-located or they may be positioned in separate locations. Multiple master assemblies may allow more than one operator to control one or more manipulator assemblies in various combinations.

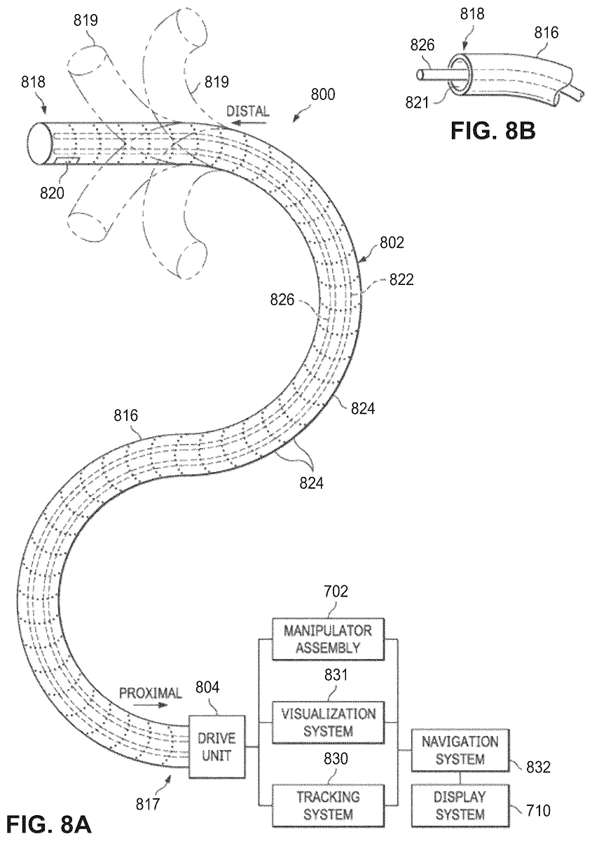

FIG. 8A is a simplified diagram of a medical instrument system 800 according to some examples. The medical instrument system 800 includes a flexible elongate device 802 (e.g., device 140, 240, 340, and/or 440), also referred to as elongate device 802, a drive unit 804, and a medical tool 826 that collectively is an example of a medical instrument 704 of a medical system 700. The medical system 700 may be a teleoperated system, a non-teleoperated system, or a hybrid teleoperated and non-teleoperated system, as explained with reference to FIG. 7. A visualization system 831, tracking system 830, and navigation system 832 are also shown in FIG. 8A and are example components of the control system 712 of the medical system 700. In some examples, the medical instrument system 800 may be used for non-teleoperational exploratory procedures or in procedures involving traditional manually operated medical instruments, such as endoscopy. The medical instrument system 800 may be used to gather (e.g., measure) a set of data points corresponding to locations within anatomic passageways of a patient, such as patient P.

The elongate device 802 is coupled to the drive unit 804. The elongate device 802 includes a channel 821 through which the medical tool 826 may be inserted. The elongate device 802 navigates within patient anatomy to deliver the medical tool 826 to a procedural site. The elongate device 802 includes a flexible body 816 having a proximal end 817 and a distal end 818. In some examples, the flexible body 816 may have an approximately 3 mm outer diameter. Other flexible body outer diameters may be larger or smaller.

Medical instrument system 800 may include the tracking system 830 for determining the position, orientation, speed, velocity, pose, and/or shape of the flexible body 816 at the distal end 818 and/or of one or more segments 824 along flexible body 816, as will be described in further detail below. The tracking system 830 may include one or more sensors and/or imaging devices (e.g., imaging unit 110). The flexible body 816, such as the length between the distal end 818 and the proximal end 817, may include multiple segments 824. The tracking system 830 may be implemented using hardware, firmware, software, or a combination thereof. In some examples, the tracking system 830 is part of control system 712 shown in FIG. 7. The tracking system 830 may implement at least some of the techniques described with reference to FIGS. 1A-6, and, to that end, may include at least portions of or be in communicative connection with the processing unit 120 of FIG. 1A.

Tracking system 830 may track the distal end 818 and/or one or more of the segments 824 of the flexible body 816 using a shape sensor 822. The shape sensor 822 may include an optical fiber aligned with the flexible body 816 (e.g., provided within an interior channel of the flexibly body 816 or mounted externally along the flexible body 816). In some examples, the optical fiber may have a diameter of approximately 800 μm. In other examples, the diameter may be larger or smaller. The optical fiber of the shape sensor 822 may form a fiber optic bend sensor for determining the shape of flexible body 816. Optical fibers including Fiber Bragg Gratings (FBGs) may be used to provide strain measurements in structures in one or more dimensions. Various systems and methods for monitoring the shape and relative position of an optical fiber in three dimensions, which may be applicable in some examples, are described in U.S. Patent Application Publication No. 2006/0013523 (filed Jul. 13, 2005 and titled “Fiber optic position and shape sensing device and method relating thereto”); U.S. Pat. No. 7,772,541 (filed on Mar. 12, 2008 and titled “Fiber Optic Position and/or Shape Sensing Based on Rayleigh Scatter”); and U.S. Pat. No. 8,773,650 (filed on Sept. 2, 2010 and titled “Optical Position and/or Shape Sensing”), which are all incorporated by reference herein in their entireties. Sensors in some examples may employ other suitable strain sensing techniques, such as Rayleigh scattering, Raman scattering, Brillouin scattering, and Fluorescence scattering.

In some examples, the shape of the flexible body 816 may be determined using other techniques. For example, a history of the position and/or pose of the distal end 818 of the flexible body 816 can be used to reconstruct the shape of flexible body 816 over an interval of time (e.g., as the flexible body 816 is advanced or retracted within a patient anatomy). In some examples, the tracking system 830 may alternatively and/or additionally track the distal end 818 of the flexible body 816 using a position sensor system 820. Position sensor system 820 may be a component of an EM sensor system with the position sensor system 820 including one or more position sensors. Although the position sensor system 820 is shown as being near the distal end 818 of the flexible body 816 to track the distal end 818, the number and location of the position sensors of the position sensor system 820 may vary to track different regions along the flexible body 816. In one example, the position sensors include conductive coils that may be subjected to an externally generated electromagnetic field. Each coil of position sensor system 820 may produce an induced electrical signal having characteristics that depend on the position and orientation of the coil relative to the externally generated electromagnetic field. The position sensor system 820 may measure one or more position coordinates and/or one or more orientation angles associated with one or more portions of flexible body 816. In some examples, the position sensor system 820 may be configured and positioned to measure six degrees of freedom, e.g., three position coordinates X, Y, Z and three orientation angles indicating pitch, yaw, and roll of a base point. In some examples, the position sensor system 820 may be configured and positioned to measure five degrees of freedom, e.g., three position coordinates X, Y, Z and two orientation angles indicating pitch and yaw of a base point. Further description of a position sensor system, which may be applicable in some examples, is provided in U.S. Pat. No. 6,380,732 (filed Aug. 11, 1999 and titled “Six-Degree of Freedom Tracking System Having a Passive Transponder on the Object Being Tracked”), which is incorporated by reference herein in its entirety.

In some examples, the tracking system 830 may alternately and/or additionally rely on a collection of pose, position, and/or orientation data stored for a point of an elongate device 802 and/or medical tool 826 captured during one or more cycles of alternating motion, such as breathing. This stored data may be used to develop shape information about the flexible body 816. In some examples, a series of position sensors (not shown), such as EM sensors like the sensors in position sensor 820 or some other type of position sensors may be positioned along the flexible body 816 and used for shape sensing. In some examples, a history of data from one or more of these position sensors taken during a procedure may be used to represent the shape of elongate device 802, particularly if an anatomic passageway is generally static.

FIG. 8B is a simplified diagram of the medical tool 826 within the elongate device 802 according to some examples. The flexible body 816 of the elongate device 802 may include the channel 821 sized and shaped to receive the medical tool 826. In some examples, the medical tool 826 may be used for procedures such as diagnostics, imaging, surgery, biopsy, ablation, illumination, irrigation, suction, electroporation, etc. Medical tool 826 can be deployed through channel 821 of flexible body 816 and operated at a procedural site within the anatomy. Medical instrument 826 may be, for example, an image capture probe, a biopsy tool (e.g., a needle, grasper, brush, etc.), an ablation tool (e.g., a laser ablation tool, radio frequency (RF) ablation tool, cryoablation tool, thermal ablation tool, heated liquid ablation tool, etc.), an electroporation tool, and/or another surgical, diagnostic, or therapeutic tool. In some examples, the medical tool 826 may include an end effector having a single working member such as a scalpel, a blunt blade, an optical fiber, an electrode, and/or the like. Other end types of end effectors may include, for example, forceps, graspers, scissors, staplers, clip appliers, and/or the like. Other end effectors may further include electrically activated end effectors such as electrosurgical electrodes, transducers, sensors, and/or the like.

The medical tool 826 may be a biopsy tool used to remove sample tissue or a sampling of cells from a target anatomic location. In some examples, the biopsy tool is a flexible needle. The biopsy tool may further include a sheath that can surround the flexible needle to protect the needle and interior surface of the channel 821 when the biopsy tool is within the channel 821. The medical tool 826 may be an image capture probe that includes a distal portion with a stereoscopic or monoscopic camera that may be placed at or near the distal end 818 of flexible body 816 for capturing images (e.g., still or video images). The captured images may be processed by the visualization system 831 for display and/or provided to the tracking system 830 to support tracking of the distal end 818 of the flexible body 816 and/or one or more of the segments 824 of the flexible body 816. The image capture probe may include a cable for transmitting the captured image data that is coupled to an imaging device at the distal portion of the image capture probe. In some examples, the image capture probe may include a fiber-optic bundle, such as a fiberscope, that couples to a more proximal imaging device of the visualization system 831. The image capture probe may be single-spectral or multi-spectral, for example, capturing image data in one or more of the visible, near-infrared, infrared, and/or ultraviolet spectrums. The image capture probe may also include one or more light emitters that provide illumination to facilitate image capture. In some examples, the image capture probe may use ultrasound, x-ray, fluoroscopy, CT, MRI, or other types of imaging technology.

In some examples, the image capture probe is inserted within the flexible body 816 of the elongate device 802 to facilitate visual navigation of the elongate device 802 to a procedural site and then is replaced within the flexible body 816 with another type of medical tool 826 that performs the procedure. In some examples, the image capture probe may be within the flexible body 816 of the elongate device 802 along with another type of medical tool 826 to facilitate simultaneous image capture and tissue intervention, such as within the same channel 821 or in separate channels. A medical tool 826 may be advanced from the opening of the channel 821 to perform the procedure (or some other functionality) and then retracted back into the channel 821 when the procedure is complete. The medical tool 826 may be removed from the proximal end 817 of the flexible body 816 or from another optional instrument port (not shown) along flexible body 816.

In some examples, the elongate device 802 may include integrated imaging capability rather than utilize a removable image capture probe. For example, the imaging device (or fiber-optic bundle) and the light emitters may be located at the distal end 818 of the elongate device 802. The flexible body 815 may include one or more dedicated channels that carry the cable(s) and/or optical fiber(s) between the distal end 818 and the visualization system 831. Here, the medical instrument system 800 can perform simultaneous imaging and tool operations.

In some examples, the medical tool 826 is capable of controllable articulation. The medical tool 826 may house cables (which may also be referred to as pull wires), linkages, or other actuation controls (not shown) that extend between its proximal and distal ends to controllably bend the distal end of medical tool 826, such as discussed herein for the flexible elongate device 802. The medical tool 826 may be coupled to a drive unit 804 and the manipulator assembly 702. In these examples, the elongate device 802 may be excluded from the medical instrument system 800 or may be a flexible device that does not have controllable articulation. Steerable instruments or tools, applicable in some examples, are further described in detail in U.S. Pat. No. 7,316,681 (filed on Oct. 4, 2005 and titled “Articulated Surgical Instrument for Performing Minimally Invasive Surgery with Enhanced Dexterity and Sensitivity”) and U.S. Pat. No. 9,259,274 (filed Sept. 30, 2008 and titled “Passive Preload and Capstan Drive for Surgical Instruments”), which are incorporated by reference herein in their entireties.

The flexible body 816 of the elongate device 802 may also or alternatively house cables, linkages, or other steering controls (not shown) that extend between the drive unit 804 and the distal end 818 to controllably bend the distal end 818 as shown, for example, by broken dashed line depictions 819 of the distal end 818 in FIG. 2A. In some examples, at least four cables are used to provide independent up-down steering to control a pitch of the distal end 818 and left-right steering to control a yaw of the distal end 881. In these examples, the flexible elongate device 802 may be a steerable catheter. Examples of steerable catheters, applicable in some examples, are described in detail in PCT Publication WO 2019/018736 (published Jan. 24, 2019 and titled “Flexible Elongate Device Systems and Methods”), which is incorporated by reference herein in its entirety.

In examples where the elongate device 802 and/or medical tool 826 are actuated by a teleoperational assembly (e.g., the manipulator assembly 702), the drive unit 804 may include drive inputs that removably couple to and receive power from drive elements, such as actuators, of the teleoperational assembly. In some examples, the elongate device 802 and/or medical tool 826 may include gripping features, manual actuators, or other components for manually controlling the motion of the elongate device 802 and/or medical tool 826. The elongate device 802 may be steerable or, alternatively, the elongate device 802 may be non-steerable with no integrated mechanism for operator control of the bending of distal end 818. In some examples, one or more channels 821 (which may also be referred to as lumens), through which medical tools 826 can be deployed and used at a target anatomical location, may be defined by the interior walls of the flexible body 816 of the elongate device 802.

In some examples, the medical instrument system 800 (e.g., the elongate device 802 or medical tool 826) may include a flexible bronchial instrument, such as a bronchoscope or bronchial catheter, for use in examination, diagnosis, biopsy, and/or treatment of a lung. The medical instrument system 800 may also be suited for navigation and treatment of other tissues, via natural or surgically created connected passageways, in any of a variety of anatomic systems, including the colon, the intestines, the kidneys and kidney calices, the brain, the heart, the circulatory system including vasculature, and/or the like.

The information from the tracking system 830 may be sent to the navigation system 832, where the information may be combined with information from the visualization system 831 and/or pre-operatively obtained models to provide the physician, clinician, surgeon, or other operator with real-time position information. The tracking system 830, the navigation system 832, and the visualization system 831 may cooperatively implement, at least partially, the functionality of the system 100 in implementing the techniques described with reference to FIGS. 1A-6. In some examples, the real-time position information may be displayed on the display system 710 for use in the control of the medical instrument system 800. In some examples, the navigation system 832 may utilize the position information as feedback for positioning medical instrument system 800. Various systems for using fiber optic sensors to register and display a surgical instrument with surgical images, applicable in some examples, are provided in U.S. Pat. No. 8,900,131 (filed May 13, 2011 and titled “Medical System Providing Dynamic Registration of a Model of an Anatomic Structure for Image-Guided Surgery”), which is incorporated by reference herein in its entirety.

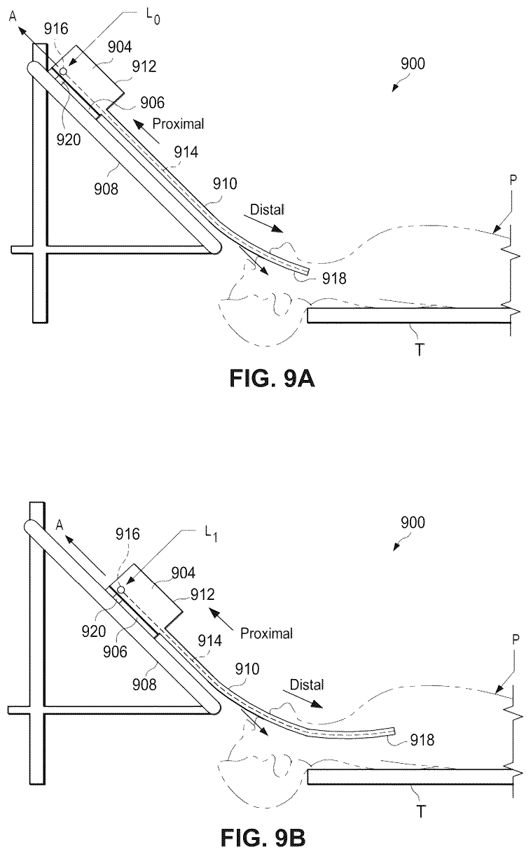

FIGS. 9A and 9B are simplified diagrams of side views of a patient coordinate space including a medical instrument mounted on an insertion assembly according to some examples. As shown in FIGS. 9A and 9B, a surgical environment 900 may include the patient P positioned on the patient table T. Patient P may be stationary within the surgical environment 900 in the sense that gross patient movement is limited by sedation, restraint, and/or other means. Cyclic anatomic motion, including respiration and cardiac motion, of patient P may continue. Within surgical environment 900, a medical instrument 904 is used to perform a medical procedure which may include, for example, surgery, biopsy, ablation, illumination, irrigation, suction, or electroporation. The medical instrument 904 may also be used to perform other types of procedures, such as a registration procedure to associate the position, orientation, and/or pose data captured by the sensor system 708 to a desired (e.g., anatomical or system) reference frame. The medical instrument 904 may be, for example, the medical instrument 704. In some examples, the medical instrument 904 may include an elongate device 910 (e.g., a catheter) coupled to an instrument body 912. Elongate device 910 may be the elongate device 140 of FIG. 1. Elongate device 910 includes one or more channels sized and shaped to receive a medical tool.

Elongate device 910 may also include one or more sensors (e.g., components of the sensor system 708). In some examples, a shape sensor 914 may be fixed at a proximal point 916 on the instrument body 912. The proximal point 916 of the shape sensor 914 may be movable with the instrument body 912, and the location of the proximal point 916 with respect to a desired reference frame may be known (e.g., via a tracking sensor or other tracking device). The shape sensor 914 may measure a shape from the proximal point 916 to another point, such as a distal end 918 of the elongate device 910. The shape sensor 914 may be aligned with the elongate device 910 (e.g., provided within an interior channel or mounted externally). In some examples, the shape sensor 914 may optical fibers used to generate shape information for the elongate device 910.

In some examples, position sensors (e.g., EM sensors) may be incorporated into the medical instrument 904. A series of position sensors may be positioned along the flexible elongate device 910 and used for shape sensing. Position sensors may be used alternatively to the shape sensor 914 or with the shape sensor 914, such as to improve the accuracy of shape sensing or to verify shape information.

Elongate device 910 may house cables, linkages, or other steering controls that extend between the instrument body 912 and the distal end 918 to controllably bend the distal end 918. In some examples, at least four cables are used to provide independent up-down steering to control a pitch of distal end 918 and left-right steering to control a yaw of distal end 918. The instrument body 912 may include drive inputs that removably couple to and receive power from drive elements, such as actuators, of a manipulator assembly.

The instrument body 912 may be coupled to an instrument carriage 906. The instrument carriage 906 may be mounted to an insertion stage 908 that is fixed within the surgical environment 900. Alternatively, the insertion stage 908 may be movable but have a known location (e.g., via a tracking sensor or other tracking device) within surgical environment 900. Instrument carriage 906 may be a component of a manipulator assembly (e.g., manipulator assembly 702) that couples to the medical instrument 904 to control insertion motion (e.g., motion along an insertion axis A) and/or motion of the distal end 918 of the elongate device 910 in multiple directions, such as yaw, pitch, and/or roll. The instrument carriage 906 or insertion stage 908 may include actuators, such as servomotors, that control motion of instrument carriage 906 along the insertion stage 908.

A sensor device 920, which may be a component of the sensor system 708, may provide information about the position of the instrument body 912 as it moves relative to the insertion stage 908 along the insertion axis A. The sensor device 920 may include one or more resolvers, encoders, potentiometers, and/or other sensors that measure the rotation and/or orientation of the actuators controlling the motion of the instrument carriage 906, thus indicating the motion of the instrument body 912. In some examples, the insertion stage 908 has a linear track as shown in FIGS. 9A and 9B. In some examples, the insertion stage 908 may have curved track or have a combination of curved and linear track sections.

FIG. 9A shows the instrument body 912 and the instrument carriage 906 in a retracted position along the insertion stage 908. In this retracted position, the proximal point 916 is at a position L0 on the insertion axis A. The location of the proximal point 916 may be set to a zero value and/or other reference value to provide a base reference (e.g., corresponding to the origin of a desired reference frame) to describe the position of the instrument carriage 906 along the insertion stage 908. In the retracted position, the distal end 918 of the elongate device 910 may be positioned just inside an entry orifice of patient P. Also in the retracted position, the data captured by the sensor device 920 may be set to a zero value and/or other reference value (e.g., I=0). In FIG. 9B, the instrument body 912 and the instrument carriage 906 have advanced along the linear track of insertion stage 908, and the distal end 918 of the elongate device 910 has advanced into patient P. In this advanced position, the proximal point 916 is at a position L1 on the insertion axis A. In some examples, the rotation and/or orientation of the actuators measured by the sensor device 920 indicating movement of the instrument carriage 906 along the insertion stage 908 and/or one or more position sensors associated with instrument carriage 906 and/or the insertion stage 908 may be used to determine the position L1 of the proximal point 916 relative to the position L0. In some examples, the position L1 may further be used as an indicator of the distance or insertion depth to which the distal end 918 of the elongate device 910 is inserted into the passageway(s) of the anatomy of patient P.

One or more components of the examples discussed in this disclosure, such as control system 712, may be implemented in software for execution on one or more processors of a computer system. The software may include code that when executed by the one or more processors, configures the one or more processors to perform various functionalities as discussed herein. The code may be stored in a non-transitory computer readable storage medium (e.g., a memory, magnetic storage, optical storage, solid-state storage, etc.). The computer readable storage medium may be part of a computer readable storage device, such as an electronic circuit, a semiconductor device, a semiconductor memory device, a read only memory (ROM), a flash memory, an erasable programmable read only memory (EPROM); a floppy diskette, a CD-ROM, an optical disk, a hard disk, or other storage device. The code may be downloaded via computer networks such as the Internet, Intranet, etc. for storage on the computer readable storage medium. The code may be executed by any of a wide variety of centralized or distributed data processing architectures. The programmed instructions of the code may be implemented as a number of separate programs or subroutines, or they may be integrated into a number of other aspects of the systems described herein. The components of the computing systems discussed herein may be connected using wired and/or wireless connections. In some examples, the wireless connections may use wireless communication protocols such as Bluetooth, near-field communication (NFC), Infrared Data Association (IrDA), home radio frequency (HomeRF), IEEE 802.11, Digital Enhanced Cordless Telecommunications (DECT), and wireless medical telemetry service (WMTS).

Various general-purpose computer systems may be used to perform one or more processes, methods, or functionalities described herein. Additionally or alternatively, various specialized computer systems may be used to perform one or more processes, methods, or functionalities described herein. In addition, a variety of programming languages may be used to implement one or more of the processes, methods, or functionalities described herein.

While certain examples and examples have been described above and shown in the accompanying drawings, it is to be understood that such examples and examples are merely illustrative and are not limited to the specific constructions and arrangements shown and described, since various other alternatives, modifications, and equivalents will be appreciated by those with ordinary skill in the art.

1. A method of localizing a flexible elongate device disposed within an anatomical structure, the method comprising:
obtaining, by one or more processors, one or more images of the flexible elongate device disposed within an anatomical structure;
determining, by the one or more processors, that a plurality of pixels or voxels within the one or more images corresponds to edges of the flexible elongate device; and
causing, by the one or more processors, a display device to display a graphical user interface depicting the flexible elongate device based at least in part on the plurality of pixels or voxels corresponding to the edges of the flexible elongate device.

2. The method of claim 1, wherein determining that the plurality of pixels or voxels within the one or more images corresponds to the edges of the flexible elongate device includes performing Sobel edge detection.

3. The method of claim 1, wherein determining that the plurality of pixels or voxels within the one or more images corresponds to the edges of the flexible elongate device includes performing a thresholding operation.

4. The method of claim 3, wherein the thresholding operation comprises a binary thresholding operation.

5. The method of claim 3, wherein the thresholding operation is based at least in part on an image histogram.

6. (canceled)

7. (canceled)

8. The method of claim 1, wherein
the one or more images comprise one or more three-dimensional (3D) images; and
the method further comprises performing a projection operation to generate a 2D image based on the one or more 3D images.

9. The method of claim 8, wherein performing the projection operation includes selecting a projection angle.

10. The method of claim 8, further comprising:
performing a de-projection operation to add a third coordinate based on two coordinates of a pixel in a 2D image, and wherein
causing the display device to display the graphical user interface depicting the flexible elongate device is based at least in part on the de-projection operation.

11. The method of claim 1, further comprising:
performing a contour-detection operation, and wherein
determining that the plurality of pixels or voxels within the one or more images corresponds to the edges of the flexible elongate device is based at least in part on the contour-detection operation.

12. The method of claim 11, further comprising:
discarding pixels associated with a contour extent below a contour extent threshold.

13. The method of claim 1, further comprising:
performing a clustering operation, and wherein
determining that a plurality of pixels or voxels within the one or more images corresponds to edges of the flexible elongate device is based at least in part on the clustering operation.

14. (canceled)

15. The method of claim 1, wherein causing a display device to display a graphical user interface depicting the flexible elongate device includes depicting only a tip portion of the flexible elongate device.

16. The method of claim 1, further comprising:
generating a curve representative of a center line of the flexible elongate device based at least in part on the plurality of pixels or voxels corresponding to the edges of the flexible elongate device, and wherein
causing the display device to display the graphical user interface depicting the flexible elongate device includes displaying at least a portion of the curve representative of the center line of the flexible elongate device.

17. The method of claim 16, wherein generating the curve representative of a center line of the flexible elongate device includes fitting at least a portion of the plurality of pixels or voxels corresponding to the edges of the flexible elongate device with a polynomial function.

18. A system comprising:
one or more processors;
a display device; and
one or more non-transitory, computer-readable media storing instructions that, when executed by the one or more processors, cause the one or more processors to:
obtain one or more images of a flexible elongate device disposed within an anatomical structure;
determine that a plurality of pixels or voxels within the one or more images corresponds to edges of the flexible elongate device; and
cause the display device to display a graphical user interface depicting the flexible elongate device based at least in part on the plurality of pixels or voxels corresponding to the edges of the flexible elongate device.

19. (canceled)

20. One or more non-transitory, computer readable media storing instructions that, when executed by one or more processors, cause the one or more processors to:
obtain one or more images of a flexible elongate device disposed within an anatomical structure;
determine that a plurality of pixels or voxels within the one or more images corresponds to edges of the flexible elongate device; and
cause a display device to display a graphical user interface depicting the flexible elongate device based at least in part on the plurality of pixels or voxels corresponding to the edges of the flexible elongate device.

21. (canceled)

22. The system of claim 18, wherein:
the one or more images comprise one or more three-dimensional (3D) images; and
the instructions, when executed, further cause the one or more processors to perform a projection operation to generate a 2D image based on the one or more 3D images.

23. The system of claim 18, wherein the instructions, when executed, further cause the one or more processors to:
perform a contour-detection operation, and wherein
determine that the plurality of pixels or voxels within the one or more images corresponds to the edges of the flexible elongate device is based at least in part on the contour-detection operation.

24. The system of claim 18, wherein the instructions, when executed, further cause the one or more processors to:
perform a clustering operation, and wherein
determine that a plurality of pixels or voxels within the one or more images corresponds to edges of the flexible elongate device is based at least in part on the clustering operation.

25. The system of claim 18, wherein the instructions, when executed, further cause the one or more processors to:
generate a curve representative of a center line of the flexible elongate device based at least in part on the plurality of pixels or voxels corresponding to the edges of the flexible elongate device, and wherein
cause the display device to display the graphical user interface depicting the flexible elongate device includes displaying at least a portion of the curve representative of the center line of the flexible elongate device.
